(19) United States
(12) Patent Application Publication
(10) Pub. No.: US 2001/0034885 A1
(43) Pub. Date: Oct. 25, 2001

(54) Non-human animal deficient in function of pituitary adenylate cyclase-activating polypeptide gene

(76) Inventors: Akemichi Baba, Nishinomiya-shi (JP); Toshio Matsuda, Osaka (JP); Hitoshi Hashimoto, Osaka (JP); Norihito Shintani, Osaka (JP)

Correspondence Address: ARMSTRONG,WESTERMAN, HATTORI,, MCLELAND & NAUGHTON, LLP, 1725 K STREET, NW, SUITE 1000, WASHINGTON, DC, 20006, US

(21) Appl. No.: 09/835,627

(22) Filed: Apr. 17, 2001

(30) Foreign Application Priority Data
Apr. 19, 2000 (JP) 2000-118088

(51) Int. Cl. A01K 67/027

(52) U.S. Cl. 800/18; 800/3

(57) ABSTRACT

As a means to elucidate involvement of the pituitary adenylate cyclase-activating polypeptide (hereinafter designated as PACAP) in the impairment and the degeneration of the central nervous system and the peripheral nervous system and the pathophysiology of diversified diseases, a knockout animal, in which the function of PACAP gene of the animal is defective in somatic cells and germ cells is prepared.

### BACKGROUND OF THE INVENTION

1. Field of the Invention

The present invention relates to a non-human animal with deficient in function of pituitary adenylate cyclase-activating polypeptide (hereinafter referred to as PACAP) gene. More particularly, the present invention relates to a non-human animal having chromosomes of somatic cells and germ cells with deficiency of function of PACAP gene.

2. Description of the Related Art

PACAP is a neuropeptide isolated from ovine hypothalamus on the basis of its ability to stimulate adenylate cyclase in cultured rat anterior pituitary cells. PACAP plays an important role as peptide hormone, neurotransmitter or neuromodulator in the central nervous system and the peripheral nervous system. For example, the facts that the expression of PACAP is increased in cerebral ischemic accident or that intracerebral administration of PACAP can suppress nerve cell death caused by such accident are demonstrated. Further, since expression of PACAP in the nerve cells is strongly increased as a result of impairment of peripheral nerve such as neurotmesis, PACAP is suggested to be an essential factor for maintaining survival of nerve cells and recovery of the impaired nerve function.

It is further reported that PACAP strongly stimulates glucose dependent insulin secretion from the pancreas. In addition, it is reported that PACAP can be involved in development of gastric ulcer. As described above, the possibility of involvement of PACAP in important physiological functions in tissues expressing PACAP has been reported. The mouse PACAP gene, a gene coding PACAP, has already been isolated by the present inventors, and the total primary sequence thereof has been elucidated (Yamamoto, et al., Gene, 211, 63-69 (1998)).

To elucidate what actions PACAP, a specific polypeptide, itself elicits on animals is, however, quite difficult, since the impairment and degeneration in the above central nervous system and the peripheral nervous system, and the pathophysiology of the above various diseases are actually mixture of complex factors.

### SUMMARY OF THE INVENTION

The present inventors have studied extensively to solve these problems. As a result, they have found that if a line of mutant animal, which was deficient in PACAP gene, genetically stable and obvious in genetic background, could be obtained, it would be quite useful as an experimental animal for studies on the physiological functions of PACAP itself as well as the pathophysiology of the above diversified diseases to which PACAP pertains, elucidation of the etiology and development of drugs for treatments, and completed the present invention. At present, no animal that is genetically stable with deficiency of function of PACAP gene is known.

The present inventors have generated mice deficient in PACAP to understand the in vivo functions of PACAP-dependent signalings, and discovered a new role for PACAP in the regulation of psychomotor functions, and found that dysfunction of PACAP-mediated signaling pathways can lead to the pathologic changes characteristic of certain psychiatric disorders.

An object of the present invention is to provide a non-human animal which is deficient in the function of PACAP gene.

Particularly, an object of the present invention is to provide the above non-human animal, especially mouse (hereinafter designated as PACAP knockout mouse), which has chromosomes of somatic cells and germ cells with deficiency of function of PACAP gene.

Namely, the present invention is as follows:

(1) A non-human animal having chromosomes of somatic cells and germ cells with deficiency of function of pituitary adenylate cyclase-activating polypeptide gene.

(2) The non-human animal according to the above (1), wherein the function is defective due to deficiency of a part or whole of exon 5 in the pituitary adenylate cyclase-activating polypeptide gene.

(3) The non-human animal according to the above (1), wherein the function is defective due to introducing a point mutation or inserting another gene in exon 5.

(4) The non-human animal according to the above (2), wherein a part or whole of exon 5 is deleted by substituting the part or whole of the exon 5 by another gene.

(5) The non-human animal according to the above (4), wherein the another gene is a marker gene.

(6) The non-human animal according to the above (5), wherein the marker gene is a neomycin resistant gene.

(7) The non-human animal according to the above (1), wherein the non-human animal is a mouse.

(8) A model animal for certain psychiatric disorders, wherein the animal is a non-human animal according to any one of the above (1) to (7).

The animal of the present invention which is deficient in the function of PACAP gene due to a genetically engineering technique is quite useful as an experimental animal for studies on the survival and the maintenance of disrupted nerve cells caused by cerebral ischemic impairment and neurotmesis, or the regulation of insulin secretion in the pancreas, the development of gastric ulcer, and the pathophysiology of diseases in PACAP expressing tissues, the elucidation of biological mechanism and development of drugs for treatment of these diseases.

### BRIEF DESCRIPTION OF THE DRAWING

FIG. 1 is a schematic drawing which shows a targeting disruption of the mouse PACAP gene. DT: diphtheria toxin A fragment gene, neo: neomycin resistant gene.

### PREFERRED EMBODIMENT OF THE INVENTION

In the present invention, the deficiency of the function of PACAP gene means that the structure of the gene is different from the naturally occurring structure and the gene cannot be expressed and PACAP cannot be produced. In order to obtain an animal which is artificially made deficient in the function of PACAP gene, the gene is cloned and the function of the gene is disrupted in vitro by any means, then the defective gene is transferred into the animal to make the animal itself or its progeny deficient in the function of the gene.

In the present invention, non-human animal(s) means any animals except for human, preferably mammalians, more preferably mice.

Means for inserting an external gene into an animal and expressing the gene in the individual of the animal or its progeny include implanting into the animal a host embryo obtained by any of (1) inserting a genomic DNA into a pronuclear embryonic phase of a fertilized ovum, (2) infecting the early phase embryo of the animal with the recombinant retrovirus using a genomic DNA, and (3) using a genomic DNA as a targeting vector to construct a homologous recombination and inserting the embryonic stem cells (ES cells) of the animal into the blastocyst or 8-cell embryo, thereby obtaining offspring, which is mated up with another individual to prepare F1 heterozygote variant animals and further to prepare F2 homozygote variant animals. These means have been known in the art for preparing transgenic animals.

Among the above methods, the gene transfer using ES cells is preferable, since it has advantages that the process can be divided into a process for transferring a gene into ES cells and a process for preparing chimera animals. ES cells are considered to be able to be cultured in principle in general mammalians. In mice, rats, rabbits and livestock such as porcine and bovine, studies are in progress for establishment of ES cells. In the animal species in which method using ES cells are not established, the above method (1) or (2) can be used. Since a method for gene transfer using ES cells has been established especially in mice, the present invention will be explained using mice as an example, but the present invention is not limited to this example.

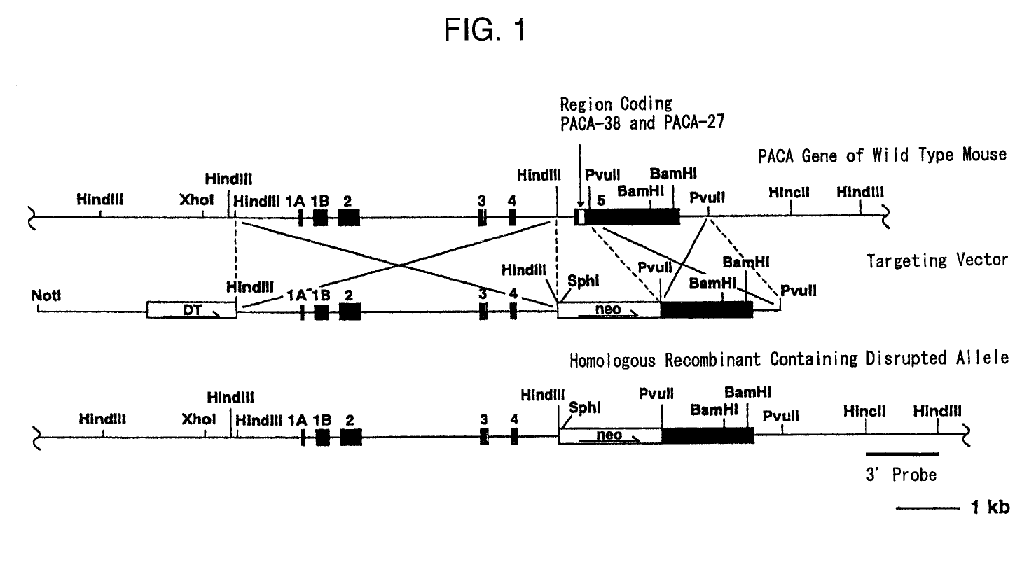

According to the description of the above reference, Yamamoto et al., Gene, 211, 63-69 (1998), the mouse PACAP gene spans 6.6 kb of the genomic DNA and is composed of six exons (exon 1A or exon 1B and exons 2-5), including two 5′-untranslated exons. Exons 1A and 1B encode the 5′-untranslated region (5′-UTR) of the PACAP DNA. Exon 2 encodes 5′-UTR, signal peptide and a part of amino-terminal peptide. Exon 3 encodes most of the amino-terminal peptide. Exon 4 encodes a PACAP related peptide (PRP). Exon 5 encodes the connecting peptide, mature PACAPs (PACAP-38 and PACAP-27), the carboxy-terminal peptide and the 3′-UTR, and contains two potential polyadenylation signals. The mouse PACAP gene encodes a PACAP precursor consisting of 175 amino acid residue in total from exon 2 to exon 5. Limiting decomposition of the precursor results in production of physiologically active PACAP-38 consisting of 38 amino acid residues or PACAP-27 consisting of 27 amino acid residues at the N-terminal side of PACAP-38. As described above, these PACAPs are encoded in a part of exon 5 in mouse PACAP gene (FIG. 1).

Consequently, if exon 5, preferably the function in the region encoding matured PACAPs on exon 5, is made defective, the mouse of the present invention which is deficient in the function of PACAP gene can be prepared. Examples of methods for preparing defective function of the region encoding matured PACAPs on exon 5 include the following: to delete a part or whole of the region, to insert a point mutation into the region, to insert another gene into the region and to substitute another gene for a part or the whole of the region to delete the above part or the whole of the region. It is preferable to select a proper marker gene as the another gene and substitute the proper marker gene for the region to construct a targeting vector (DNA for homologous recombination) for deficiency of the function of said region. At this time, the marker gene used is preferably such that a positive/negative selection can be performed in order to select the objective homologous recombinant.

A process for preparing a PACAP knockout mouse is described below.

Preparation of targeting vector of mouse PACAP gene (DNA for homologous recombinant)

In order to select the objective homologous recombinant and simultaneously make the function of PACAP gene defective, a positive/negative selection is performed. For these functional deficiency and selection, insertion of neomycin resistant gene (neo) and diphtheria toxin A fragment (DT) are especially preferable according to Yagi et al. (Yagi, Nada, Watanabe et al., Analytical Biochemistry, 214, 77-86 (1993)).

PACAP gene deficiency of ES cells by means of homologous recombination

The thus obtained DNA for the homologous recombination is suspended in a buffer such as phosphate buffer saline (PBS) containing mouse ES cells (e.g. E14 strain), and the gene is transferred into ES cells. Selective culture using G418 as a selective agent is performed for positive selection. Colonies resistant to the selective agent are examined by Southern blotting to confirm homologous recombinant. The thus obtained ES cells are injected into host embryonic cells such as blastocyst or 8-cell embryo of a mouse of a proper line, and a chimera mouse is prepared by conventional method to obtain a PACAP knockout mouse in which the homologous recombinant ES cells are transferred into the germ cell line.

### EXAMPLE 1

### Preparation of PACAP Knockout Mouse

(1) Preparation of DNA for homologous recombinant DNA in mouse PACAP genomic DNA

In order to disrupt the PACAP gene, 0.5 kb region encoding matured PACAPs on exon 5 was made defective by conventional method, and neomycin resistant gene was inserted therein and diphtheria toxin A fragment gene was inserted into the upstream of exon 1A. The homologous region with the genomic DNA was constructed to be 5.3 kb length between the diphtheria toxin A fragment gene and the neomycin resistant gene and 2.1 kb length of the downstream of the neomycin resistant gene. The thus obtained construct was inserted into pBluescript KS and split with restriction enzyme NotI for linearization at the time of transfer into ES cells, whereby a DNA for homologous recombination, a targeting vector, was obtained (FIG. 1).

(2) PACAP gene deficiency of ES cells by transferring DNA for homologous recombination

One hundred micrograms of DNA for homologous recombination was suspended in PBS (137 mM NaCl, 2.7 mM KCl, 10 mM Na₂HPO₄ and 1.8 mM KH₂PO₄) containing 3×10⁷ mouse ES cells (E14 strain), and a gene transfer was performed under the conditions of an electric field strength of 2 kV/cm and an electrostatic capacity of 3 μF. After 24 hours of the transfer, a selection culture was performed with 150 μg/ml of G418 (Geneticin, GIBCO BRL).

After 8 days of the gene transfer, G418 resistant colonies were transferred into 96 well microplate (IWAKI 3870-096) added with PBS containing 50 μl of 0.25% trypsin (GIBCO BRL) using a micro pipette, and treated for several minutes to prepare single cells by pipetting. These were transferred to 48 well plate (IWAKI 3830-048) and cultured. After 3-4 days, at the stage when cells on the 48 well plate reached confluent or were close to confluence, the cells were treated with 0.25% trypsin, then transferred to two wells on the 24 well plate (IWAKI 3820-024) and further cultured. After the cells were proliferated again, genomic DNA for Southern analysis was extracted from the cells in the one well of 24 well plate, and cells in another well were transferred into the 6 well plate (IWAKI 3810-006), cultured and then preserved in freezing.

The genomic DNA was extracted from G418 resistant cells, digested with restriction enzyme HindIII and then subjected to a Southern blotting analysis using as a probe 1.1 kb of a HincII-HindIII fragment in the downstream of exon 5. The homologous recombinant containing disrupted allele (FIG. 1) and non-homologous recombinant were confirmed by detecting bands of 6.3 kb and 5.0 kb, respectively. The number of recombinant colonies was 4 (clones 41, 44, 65-9 and 86) of 264 colonies of G418 resistant colonies.

(3) ES cells and culture thereof

As for ES cells, E14 strain derived from 129/Ola mouse blastocyst was used. For culture of ES cells, ES culture medium consisting of Glasgow minimum essential medium (GMEM, GIBCO BRL) added with 10% fetal calf serum (FCS), 0.1 mM 2-mercaptoethanol, a non-essential amino acid solution, 1 mM sodium pyruvate and LIF (GIBCO BRL).

(4) Preparation of chimera mouse using PACAP deficient ES cells

This was performed by conventional manner. After inserting the homologous recombinant clones 41 and 65-9 of ES cells into 15 blastocysts and 9 blastocysts of C57BL/6 mice, these blastocysts were implanted into uteri of recipient ICR strain, female, to obtain 4 and 2 offsprings, respectively. Among the respective delactated 2 offsprings, each one could be determined as chimera mouse by observing hair color, each of which was morphologically male. Rate of contribution of ES cells in these chimera mice was about 70-80%.

(5) Mating of chimera mice

Chimera mice obtained by the implantation were mated with C57BL/6 mice, female, and examined as to whether the obtained offsprings (F1 heterozygote mice) were originated from PACAP gene-defective ES cells or not was detected. If the germ cells of the chimera mouse are derived from the ES cells, the color of hair of the offsprings shows wild type color, and if it is derived from blastocysts of C57BL/6 mouse, the color of offsprings show black. Transfer of ES cells to germ cell line was confirmed in both of chimera mice originated from ES cell clones 41 and 65-9, respectively.

As the results of mating with chimera mouse derived from ES cell clone 41 and C57BL/6 mouse, female, total 51 offsprings were obtained from 6 times of deliveries. Among them, 45 mice survived and all showed wild color. As the results of mating with chimera mouse derived from ES cell clone 65-9 and C57BL/6 mouse, female, total 70 offsprings were obtained in 8 times of deliveries. Among them, 62 mice survived, further among the survivals, 21 mice showed wild color.

As a result of Southern blotting analysis of the wild color mice, 20 out of 45 mice derived from the clone 41, and 8 out of 21 mice derived from the clone 65-9, were confirmed to be deficient in the PACAP gene. Matings were further performed within F1 heterozygotes mice which were confirmed to be deficient in the gene, or within F2 heterozygotes mice obtained from mating between F1 heterozygote mouse and C57BL/6 mouse. As a result, 134 offsprings and 33 offsprings, obtained from clone 41 and clone 65-9, respectively, were obtained. Southern blotting analyses showed that F2 or F3 homozygote mice were deficient in the PACAP gene, in 25 mice and 4 mice from clone 41 and clone 65-9, respectively.

What is claimed is:

1. A non-human animal having chromosomes of somatic cells and germ cells with deficiency of function of pituitary adenylate cyclase-activating polypeptide gene.

2. The non-human animal according to claim 1, wherein said function is defective due to deficiency of a part or whole of exon 5 in said pituitary adenylate cyclase-activating polypeptide gene.

3. The non-human animal according to claim 1, wherein said function is defective due to introducing a point mutation or inserting another gene in exon 5.

4. The non-human animal according to claim 2, wherein a part or whole of exon 5 is deleted by substituting the part or whole of the exon 5 by another gene.

5. The non-human animal according to claim 4, wherein said another gene is a marker gene.

6. The non-human animal according to claim 5, wherein said marker gene is a neomycin resistant gene.

7. The non-human animal according to claim 1, wherein said non-human animal is a mouse.
